(19) United States
(12) Patent Application Publication
(10) Pub. No.: US 2022/0256840 A1
(43) Pub. Date: Aug. 18, 2022

(54) METHOD AND APPARATUS FOR STORAGE OF BIOLOGICAL MATERIAL

(71) Applicant: CryoStasis Ltd., Westport (CA)

(72) Inventors: Olga Kukal, Westport (CA); Thomas Furman Allen, Westport (CA); Bill Russell Alexander, Shrewsbury, MA (US)

(21) Appl. No.: 17/624,811

(22) PCT Filed: Jul. 3, 2020
(86) PCT No.: PCT/CA2020/050929

§ 371 Date: Jan. 4, 2022

Related U.S. Application Data
(63) Continuation of application No. 16/501,918, filed on Jul. 5, 2019.

(51) Int. Cl. A01N 1/02

(52) U.S. Cl. CPC A01N 1/0284; A01N 1/0289; A01N 1/021; A01N 1/0252

(57) ABSTRACT

Methods and apparatus use low temperature and elevated pressure to depress the freezing and melting temperature of water and aqueous solutions, to induce suspended animation in materials including but not limited to biological material, soluble molecules, organic and inorganic compounds. Disposing such materials in a pressure vessel and increasing the pressure to about 210 MPa depresses the freezing and melting temperature of water, biological matter, and materials in aqueous solution, to about −22° C. Storage at low temperature under high pressure suspends metabolic activity and induces cryostasis. The methods and apparatus may be used for cryo-banking biological materials that cannot be frozen or vitrified, or otherwise preserved, including, but not limited to, cells, tissues, human organs for transplantation, and entire organisms.

### RELATED APPLICATION

This application claims the benefit of the filing date of U.S. application Ser. No. 16/501,918 filed on 5 Jul. 2019, the contents of which are incorporated herein by reference in their entirety.

### FIELD

The field of the invention is long-term preservation and storage of sensitive materials that are damaged by freezing. More specifically, the invention relates to long-term preservation and storage of sensitive materials such as aqueous solutions and biological material below their freezing temperature by applying increased pressure to avoid freezing at temperatures as low as −22° C.

### BACKGROUND

In tissue or organ preservation, the current state of the art involves perfusion and storage at body temperature or in the hypothermic range of 4° C. and above. These methods are efficacious for the preservation and transport of organs over several days, but are not suitable for long-term bio-banking (i.e., weeks, months, years). There is a growing need for transplantable organs, and countless people die each year waiting for an organ transplant. This situation can be partly ameliorated by improving the preservation of organs during transport, but these advances will only be incremental. Once regenerative technologies for 3-D printing, growing, and genetic/immunological modification of organs for xenotransplantation are realized, the need for transplantable organs will present new challenges. It will be necessary to store organs from these sources until they are needed for transplantation, because the processes used to manufacture the organs will take an interval of time that might not be available to a patient in critical need. Further, individuals may wish to have a set of their own organs/tissues generated and preserved for future needs.

The effect of pressure at ambient temperature on molecules, cells and organisms has been studied with results showing that survival is possible even at ultra-high pressures. Some cells and organisms can remain viable at temperatures near absolute zero or in outer space. Over more than half a century, researchers have attempted to develop methods of freezing or vitrifying organs as a means of long-term preservation. All attempts have met with failure. There remains a need for long-term preservation of biological matter and other aqueous-based organic and inorganic materials.

### SUMMARY

One aspect of the invention relates to a method for storing/preservation, including but not limited to, water, organic and inorganic aqueous-based materials/substances/media, materials in aqueous suspension, aqueous solutions, aqueous mixtures, aqueous colloids, aqueous-based materials, biological materials, biologics, and materials of biological origin at temperatures below their freezing, i.e., melting, temperature at ambient pressure by increasing pressure. Increasing the pressure applied to any or all of the above materials, in a pressure vessel, depresses their freezing, i.e., melting temperature (point). The temperature range for storage where it is not possible for the above materials to freeze or vitrify extends from −0.001° C. to −21.985° C. The melting point, i.e., freezing point, of the above materials being depressed by pressure over the pressure range from ambient pressure to 209.9 MPa, or about 210 MPa. Such biological materials may include, but are not limited to, organic molecules, molecular complexes, nucleic acids, saccharides, amino acids, peptides, proteins, enzymes, organelles, organoids, cells, tissues, organs, and organisms.

In various embodiments, the method includes storing aqueous-based material under pressure to prevent phase transition to solid and maintain it in stable liquid state, or in a in metastable supercooled liquid state, wherein the material stored is water, or in water containing inorganic solutes in aqueous solution, or wherein the material stored is water containing organic solutes in aqueous solution, or wherein the material stored is water containing organic and inorganic solutes in aqueous solution, or wherein the material stored is water and a mixture of organic material, or wherein the material stored is water containing a colloid(s), or wherein the material stored is water in a mixture with either or both organic and/or inorganic materials, or wherein the material stored is water in a mixture with biological material(s), or the material stored is water with biological material(s) present and/or in suspension, or wherein the material stored is water containing organic and/or inorganic solutes and with biological material(s) present and/or in suspension, or wherein the material stored is water containing organic and/or inorganic solutes and colloid(s) with biological material(s) present and/or in suspension, or wherein the material stored is water in a mixture with compounds, organic and/or inorganic, and containing solutes both organic and/or inorganic, colloid(s), with biological material(s) present and/or in suspension.

In one embodiment, the invention provides a method for depressing the supercooling temperature (point) of, but not limited to, organic and inorganic aqueous-based materials/substances/media, materials in aqueous suspension, aqueous solutions, aqueous mixtures, aqueous colloids, aqueous-based materials, biological materials, and materials of biological origin at temperatures below their freezing, i.e., melting, temperature at ambient pressure by increasing the pressure applied to the material/substance and cooling to temperature(s) below their freezing/melting point at a given pressure. Accordingly the materials/substances can be supercooled and remain in a metastable liquid state over the range from −0.001° C. to −92° C. The supercooling occurs over the pressure range from ambient pressure to 209.9 MPa. The material being stored is supercooled if the storage temperature is below the pressure-depressed (pressure-determined) freezing/melting point of the material. The biological materials may be, but are not limited to, organic molecules and molecular complexes, nucleic acids, saccharides, amino acids, peptides, proteins, enzymes, biologics, organelles, organoids, cells, tissues, organs, and organisms.

In one embodiment, the invention provides a method for lowering the freezing point of the materials by further depressing the freezing temperature of the materials by the addition of solutes to storage media and material being stored, resulting in a further freezing point depression of 1.86° C. per mole of solute added; or a fraction or multiplier thereof, wherein freezing point is depressed by 1.86° C. per mole or fraction of 1.86° C. per mole fraction of solute added.

In one embodiment, the invention provides a method for lowering the freezing point of aqueous media under the conditions described herein, by further depressing the freezing temperature of the aqueous media by adjusting colligative properties by adding a mole or mole fraction of a solute or solutes to the aqueous solution, mixture, colloid or combination thereof. A further freezing point depression may be achieved by the addition of non-colligative substances, including but not limited to, antifreeze proteins, antifreeze saccharides, ice binding peptides, and other non-colligative agents that provide an additive freezing point depression by means of ice inhibiting or ice binding, thus preventing, inhibiting, controlling, and/or sequestering ice crystal growth. The media may or may not contain biological material, including but not limited to, organic molecules and molecular complexes, nucleic acids, saccharides, amino acids, peptides, proteins, enzymes, biologics, organelles, organoids, cells, tissues, organisms. In various embodiments, the antifreeze proteins may be from, for example, the meal worm beetle (Tenebrio molitor), Antarctic fish (Type I, Type III), or rye grass (Lolium perenne).

Another aspect of the invention relates to a method for storing biological material, comprising: disposing the biological material in a pressure vessel; filling the pressure vessel with a drive liquid; is placing air from the pressure vessel and sealing the pressure vessel; increasing pressure on the drive liquid using a pressure generator and decreasing temperature below 0° C. inside the pressure vessel; wherein at a selected temperature a selected pressure is applied to the drive liquid using the pressure generator whereby the drive liquid in the pressure vessel is maintained in a stable, liquid state; wherein freezing of the biological material is prevented at a storage temperature below 0° C. by applying a selected pressure to the drive liquid.

In one embodiment further comprises: disposing the biological material in a sample bag with a preservation solution; evacuating air from the sample bag; and sealing the sample bag; wherein the preservation solution and the drive liquid are maintained in a stable, liquid state.

In one embodiment decreasing the temperature and increasing the pressure comprises increasing pressure from ambient conditions at 1,000 psig/minute (6.9 MPa) in increments of 200 psig (1.4 MPa) to about 30,000 psig (210 MPa), and decreasing temperature from ambient conditions to about −22° C.

In one embodiment the biological material comprises one or more of organic molecules, molecular complexes, nucleic acids, saccharides, amino acids, peptides, proteins, enzymes, organelles, organoids, cells, tissues, organs, organisms, and an aqueous solution.

In one embodiment the preservation solution comprises water and one or more of biological material, soluble molecules, organic and/or inorganic compounds, material in aqueous suspension, aqueous solution, aqueous mixture, aqueous colloids, aqueous-based material, and material of biological origin.

In one embodiment the biological material comprises cells, tissues, organs, or entire organisms.

In one embodiment the storage temperature is about −22° C.

In one embodiment, at the storage temperature the applied pressure is about 30,000 psi (210 MPa).

In one embodiment the storage temperature and applied pressure prevent freezing and cell damage by maintaining cells a metastable supercooled liquid state.

In one embodiment the preservation solution comprises a solute. The solute may comprise one or more of antifreeze protein, ice binding protein, antifreeze saccharide, ice binding saccharide, ice binding peptide, and other non-colligative agents. The solute may prevent, inhibit, control, or sequester ice crystal growth, and/or prevent nucleation of ice.

In one embodiment the drive liquid comprises propylene glycol or ethylene glycol, oil, petroleum, fish oil, mineral oil, vegetable oil, water, seawater, any combination thereof.

In one embodiment the selected storage temperature is from about −5° C. to about −22° C.

Another aspect of the invention relates to an apparatus for storing biological material, comprising: a reservoir for housing a drive liquid; a pressure vessel having an internal well adapted for receiving the biological material, the pressure vessel operably connected to the reservoir to receive drive liquid from the reservoir; a pressure generator operably connected to the pressure vessel and the reservoir, that applies pressure on the drive liquid; a pressure transducer that provides an indication of the pressure of the drive liquid in the pressure vessel; a temperature sensor that senses temperature of the pressure vessel; and a refrigeration device adapted to provide a controlled pressure vessel internal temperature below about 0° C.; wherein at a selected pressure vessel temperature below about 0° C. the pressure generator applies a selected pressure to the drive liquid to maintain the drive liquid in the pressure vessel in a stable, liquid state.

In one embodiment the apparatus further comprises a data acquisition system (DAQ) that acquires data from one or more of the pressure transducer, the temperature sensor, the pressure generator, and the refrigeration device.

In one embodiment the apparatus further comprises a controller operably connected to one or more of the pressure transducer, the temperature sensor, the pressure generator, and the refrigeration device; wherein the controller monitors and maintains at least one of a selected internal pressure vessel temperature and a selected pressure on the drive liquid in the pressure vessel.

In one embodiment the pressure generator is automated and driven mechanically, electrically, pneumatically, or hydraulically by the controller.

In one embodiment the refrigeration device further comprises a heater. The heater may comprise a temperature sensor and temperature controller.

In one embodiment the refrigeration device comprises proportional-integral-derivative (PID) control.

In one embodiment the apparatus further comprises an evaporator.

In one embodiment the apparatus further comprises at least one valve that, when closed, allows isolation and removal of the pressure vessel from the apparatus; wherein the pressure vessel retains the applied pressure of the drive liquid when removed from the apparatus.

Another aspect of the invention relates to a pressure vessel for storing biological material, comprising: a housing having a cavity including a first portion, and a sample well that receives the biological material and a drive liquid; the first portion of the housing including an overflow channel that is open to an exterior of the housing; a lid including a first portion adapted to engage the first portion of the housing whereby a position of the lid within the housing is adjustable over a range from a first position to a closed position; the lid including a second portion adapted to partially fit into the sample well of the housing; the first portion of the lid including a port adapted to interface with external equipment; the lid including a drive liquid channel adapted to conduct drive liquid through the lid between the port and the sample well; wherein adjusting the lid to the closed position expels excess drive liquid from the sample well via the port and the overflow channel, and the second portion of the lid seals the sample well; wherein the pressure vessel is adapted to sustain an internal pressure of drive liquid in the sample well of at least about 30,000 psi (210 MPa).

In one embodiment the pressure is applied to drive liquid in the sample well by the external equipment via the port.

In one embodiment the pressure vessel further comprises at least one valve disposed between the port and the external equipment; wherein, when closed, the at least one valve isolates the pressure vessel from the external equipment and maintains an internal pressure of the sample well.

In one embodiment the overflow channel is adapted to receive a temperature sensor.

Another aspect of the invention relates to an apparatus for storing biological material at temperatures below 0° C. without freezing. In various embodiments the apparatus includes a refrigeration/heating system for cooling and/or heating a fluid, in a chamber containing a pressure vessel(s), or that flows through a series of circuits in the pressure vessel's wall(s) or is attached to the outside of a pressure vessel(s) during or after pressurization; and warms the fluid while warming the pressure vessel during or after de-pressurization; and one or more of: wherein the refrigerator and heater are separate components that are controlled either manually, electrically, electronically, or by a computer; wherein the refrigerator and heater are integrated into one component that is controlled either manually, electrically, electronically, or by a computer; wherein the refrigerator uses reverse cycle for heating and is controlled either manually, electrically, electronically, or by a computer; wherein the refrigerator and/or heater uses a piston compressor, evaporator, and condenser; wherein the refrigerator and/or heater uses a reciprocating piston compressor, evaporator, and condenser, and is controlled either manually, electrically, electronically, or by a computer; wherein the refrigerator/heater is thermoelectric and is controlled either manually, electrically, electronically, or by a computer; wherein the refrigerator/heater is a sterling refrigerator, sterling pulse tube cooler, and/or heater and is controlled either manually, electrically, electronically, or by a computer; wherein the refrigerator/heater is a sonic or ultrasonic device and is controlled either manually, electrically, electronically, or by a computer; wherein the refrigerator operates by means of evaporative cooling (e.g., liquid nitrogen, dry ice) and is controlled either manually, electrically, electronically, or by a computer; wherein heating and cooling are by radiation and are controlled either manually, electrically, electronically, or by a computer; wherein heating and cooling are by convection and are controlled either manually, electrically, electronically, or a computer; wherein heating and cooling are by induction and are controlled either manually, electrically, electronically, or by a computer; wherein resistance is used for heating and is controlled either manually, electrically, electronically, or by means a computer; wherein a laser or maser is used for heating and/or cooling and are controlled either manually, electrically, electronically, or by a computer.

In various embodiments, the apparatus includes control(s), a set of controls, a control system or systems, or a controller to initiate and/or maintain, or stop its operation; and to set and/or adjust the environment within the system as a whole and its components. In various embodiments, the temperature inside the pressure vessel can be cooled or maintained by a cooling system with a temperature controller, and the temperature inside the pressure vessel can be warmed or maintained by a heating system using a separate controller. In various embodiments, the controller for cooling and the controller for warming may be operated simultaneously, or a single temperature controller may be used to control the temperature during cooling and warming; wherein the temperature controller used during cooling can control the rate of temperature change; wherein the temperature controller used during warming can control the rate of temperature change. In one embodiment a single controller may be used to control cooling and warming and the rate of cooling and warming. In one embodiment a separate controller may be used during pressurization to control the rate of pressurization or pressurize ballistically. In one embodiment a controller may be used during pressurization to control the rate of de-pressurization or de-pressurize ballistically. In one embodiment a single controller may be used to control pressurization and de-pressurization and the rate of pressurization and de-pressurization. In one embodiment a single controller may be used to control temperature during warming and cooling, and the rate thereof; it can also control pressurization and de-pressurization, and the rate thereof. Any or all of the aforesaid control devices both for pressure and for temperature, or individually, may be mechanical, electrical, electronic, or computer. Any or all of such control devices can control by a set point, rate of change, and/or duration at set point for either or both temperature and pressure. Controller(s) may have a temperature sensor that provides the controller with the current temperature inside the refrigerator and/or pressure vessel. The controller(s) may have a pressure sensor, transducer, and/or gauge that provides the controller with the current pressure inside the pressure vessel, piping system or parts thereof.

In one embodiment, the apparatus provides a device that monitors temperature, by reading and/or recording the temperature inside the refrigerator/heater, inside the pressure vessel, inside the wall of the pressure vessel, or from the surface of the pressure vessel, in real time. Temperature readings, either analog or digital, may be taken automatically at intervals, or manually at intervals, the readings may be recorded manually, mechanically, electrically, electronically, or by a computer(s). Temperature readings are provided by sensors such as thermometer(s), thermistor(s), resistance thermal device(s) (RTD), thermocouple(s), infra-red sensor(s), infra-red camera(s), pyrometer(s), spring thermometer(s), liquid in a column thermometer(s), or any other mechanical, chemical, liquid crystal, electrical, or electronic sensor(s). Data from any and/or all of the temperature sensors listed above may be used as input temperature information for the controller(s) and control(s) in above embodiments.

In one embodiment, the apparatus provides a device that monitors pressure, by reading and/or recording pressure inside the pressure vessel, and/or in or from the pressure generator, and/or inside part(s) or all of the piping system. Pressure readings are produced from pressure transducer(s), analog pressure gauge(s), and displayed in real time on analog and/or digital gauge(s). Data from the pressure gauge(s) or pressure transducer(s) may be recorded mechanically, electrically, electronically, or using computer(s). Data from any and/or all pressure sensors listed above may be used as pressure information for the controller(s) and control(s) in above embodiments.

### BRIEF DESCRIPTION OF THE DRAWINGS

For a better understanding of the invention and to show more clearly how it may be carried into effect, reference will now be made, by way of example, to the accompanying drawings, wherein:

FIG. 1 is a phase diagram of water showing pressure/temperature values at which water remains in a stable, liquid state, including the lowest temperature and corresponding pressure at which water is in a stable, liquid form (designated as “A”).

FIG. 2 depicts one embodiment of an apparatus for preservation of biological material.

FIG. 3 depicts an expanded view of one embodiment of a pressure vessel for containing biological material during long-term preservation.

FIGS. 4A-4E are schematic diagrams depicting assembly of a pressure vessel, according to one embodiment.

FIGS. 5A and 5B are plots showing pressure and temperature curves for placing biological material into storage, and recovering the biological material from storage, respectively, according to one embodiment.

### DETAILED DESCRIPTION OF EMBODIMENTS

### Definitions

“Stasis” or “cryostasis” as used herein is used to describe a state of suspended metabolic and molecular activity. Cryostasis pertains more specifically to the sub-zero ° C. temperature and pressure range described herein.

“Suspended animation” pertains to a state of inactivity similar to that described above.

“Material”, “substance”, “matter” are terms used interchangeably in the description. They refer to either biological or inorganic constituents that are difficult to preserve over long-term period.

“Biological material” refers to carbon-containing, living matter or previously viable matter, or components thereof, including but not limited to molecules, proteins, cells, organelles, organoids, tissues, organs, organisms.

“Aqueous-based material” is a general term for any organic or inorganic matter that is soluble in water, or suspended in water, or contains water.

“Sub-zero” temperature is used in reference to storage at any temperature below 0° C.

“Banking” or “bio-banking” applies to the long-term preservation and storage of either biological or inorganic material.

“Storage” and “preservation” are terms used interchangeably throughout the description, and refer to the conservation and maintenance of material in cryostasis.

The term “˜” as used herein refers to the following numbers being approximate and not limited to the precise numeral stated.

The singular forms “a”, “an” and “the” include plural referents unless clearly stated otherwise.

“Fluid” refers to a gas, liquid, or a combination thereof, unless clearly specified.

“Supercooled” or “undercooled” refers to the metastable state of water below its melting temperature of 0° C. and atmospheric pressure.

“Colligative” depression of the melting (freezing) temperature of water is defined by the number of molecules in solution. One mole of solute dissolved in 1 litre of water results in 1.86° C. melting point depression.

“Non-colligative” depression of the melting (freezing) temperature of water is achieved through ice inhibiting or ice binding agents that prevent, inhibit, control, and/or sequester ice crystal growth.

“Long-term” as pertains to this invention refers to any time period from days, weeks, months, and years, unless specifically stated.

“Freezing point depression” (FPD) refers to the lowering of melting (freezing) temperature of water below 0° C. It can be achieved as described in this document through increase in pressure, supercooling, and/or addition of colligative or non-colligative acting substance.

As used herein, the term “UW® solution”, also known as University of Wisconsin Solution, refers to a preservation solution (Southard, J. H. et al., Transplantation Reviews 7(4): 176-190, 1993).

### EMBODIMENTS

Preservation of aqueous-based substances and biological materials sensitive to cryoinjury and/or freezing has been a dilemma. Some molecules (e.g., DNA), cells (e.g., bovine spermatozoa) and organisms (e.g., tardigrades, brine shrimp) can be successfully stored frozen for years. However, most biological substances (e.g., mammalian organs) cannot survive freezing or long-term storage. The reasons for this are multifold and relatively well understood. For instance, the ˜9% increase in volume during phase change from liquid to solid water (Ice Ih) causes physical damage to membranes, cells and molecular machinery. This damage is exacerbated by cell dehydration as a result of osmotic imbalance, and recrystallization of ice during the thawing process. Rapid, uniform rates of freezing are difficult, if not impossible, to achieve for biological substances that have volumes that are numerically (dimentionless) greater than their surface areas, such as human organs. In these cases, freezing starts rapidly from the outside, and when the interior later freezes, it expands and ruptures the exterior layers, hence causing physical damage. Embodiments described herein are aimed to circumvent inherent problems with phase change between liquid and solid by preventing it. An apparatus and method, as described herein, has been devised to prevent phase change, where water and aqueous-based substances can be maintained over long-term intervals in a stable, liquid state, at temperatures below their melting point at atmospheric pressures by means of increased pressure.

Embodiments described herein address the need for long-term storage and preservation of organs and other biological materials. They are also suitable for, but not limited to, long-term preservation of organic molecules, proteins, organelles, organoids, cells, tissues, organs, biologics, pharmaceuticals, and early studies indicate that it could be used to store entire organisms in a state of suspended animation, possibly facilitating interstellar travel. It has been documented that some molecules, cells and even organisms can tolerate extreme environmental conditions.

Embodiments described herein provide methods for preservation of aqueous-based substances at low temperatures using elevated pressure to depress the freezing/melting temperature of water and/or aqueous substances. According to embodiments, pressure is applied to the aqueous substances, biological materials, etc. using a pressure generator over the range of low temperatures used for preservation/storage. Initial application is the long-term storage, bio-banking, of human organs for transplantation. Embodiments provide apparatus for storing biological materials according to tthe methods described herein.

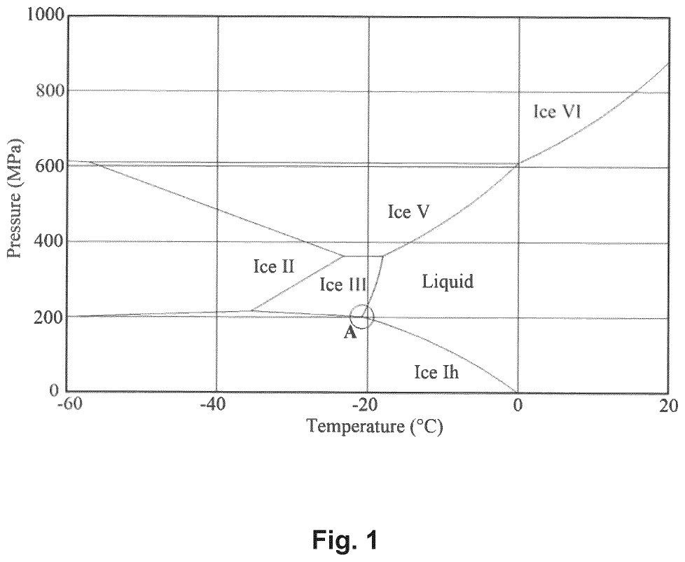

The objective of embodiments described herein is to provide a solution to the problem of long-term preservation of biological materials, such as human organs. The solution is to avoid freezing (phase change) and maintain sensitive (i.e., unfreezable) materials in a stable, liquid state at the lowest attainable temperature. This is achieved by applying pressure to the aqueous substances, biological materials, etc. using a pressure generator over the range of low temperatures used for preservation/storage. Embodiments described herein induce a state of molecular/physiological “stasis”, through the applied elevated pressure, to depress the freezing temperature (i.e., melting temperature) of water, biological matter, and other aqueous-based materials, both organic and inorganic. “Stasis” as it pertains to this invention is defined as “cryostasis”, a more accurate term, due to the low temperatures required to induce this state. Embodiments described herein employ pressure and temperature in concert, facilitate long-term preservation (e.g., months, years) in cryostasis, and provide a means of bio-banking. The pressures involved can also induce a metastable, supercooled state that may be used for long-term preservation of aqueous-based materials. Embodiments described herein utilize physicochemical properties of water, and its interactions with pressure and temperature, to maintain aqueous-based materials in a stable, liquid state. One embodiment provides preservation at the lowest temperature and corresponding pressure at which water is in a stable, liquid state, with no possibility of freezing (see FIG. 1). At pressures to achieve the freezing/melting temperature depression, molecular motion and metabolism is suppressed, resulting in cryostasis.

The invention is based, at least in part, on the hypothesis: the colder biological and other aqueous-based materials are stored without freezing and thawing (i.e., without a phase transition), the longer they will remain in usable (functional) condition (i.e., the lower the storage temperature, the longer the viable storage duration). Hence the question arises: Can temperature of living matter be lowered sufficiently without freezing in order to induce cryostasis? For example, mammalian cells, tissues, organs, and organisms are aqueous-based with approximately 300 millimoles of dissolved solutes. Based on colligative properties, these 300 millimoles of solutes result in a 0.55° C. freezing point depression of the solution within mammalian tissues. A storage temperature of −0.55° C. is not low enough to sufficiently extend (i.e., only by hours, not even days) the usable life of an organ for transplantation. In order to achieve storage at temperatures low enough to preserve cells, tissues, organs, and organisms for months or years an alternative methodology is needed. The key to this methodology lies in the relationship of temperature to pressure.

Embodiments use elevated pressure (i.e., above ambient, atmospheric pressure) to depress the freezing/melting point of water and aqueous solutions. The freezing point of pure water, and thus all aqueous-based and biological material, can be depressed by ˜1° C. per ˜9.5 MPa (Daucik, K. et al., The International Association for the Properties of Water and Steam, IAPWS R14-08, 2011). For instance, pressure of −210 MPa lowers the freezing point of water and aqueous solutions to ˜−22° C. Under these environmental conditions, molecular motion is reduced to the point that metabolic function is suppressed, resulting in a state of suspended animation, which is referred to herein as “cryostasis”. As described herein, cells, tissues, organs, and organisms stored under high pressure/low temperature conditions for days to weeks to months do not show signs of deterioration, apoptosis or necrosis and retain their functionality (see Table 1). A limit of the maximum storage interval is yet to be determined under these described environmental conditions for biological substances and other aqueous-based materials and may well have no tangible temporal limit.

Broadly stated, embodiments described herein provide for storing biological and aqueous-based materials, unfrozen below 0° C., by applying pressure elevated above ambient pressure. Biological and aqueous-based substances stored under ˜210 MPa of pressure and at a temperature no lower than ˜−22° C. will remain in a stable liquid state, because as pressure increases, the melting/freezing point of water decreases. FIG. 1 is a phase diagram of water that depicts the relationship of pressure and temperature and the fusion curve (solid-liquid boundary) that delineates at which pressure/temperature values water remains in a stable, liquid state. Some embodiments focus on the lowest temperature and corresponding pressure at which water is in a stable, liquid form. At temperatures below this lowest temperature and its corresponding pressure, water either supercools (undercools) or forms Ice III or Ice Ih (see FIG. 1, “A”). Likewise, at pressures above the pressure corresponding to the lowest temperature for stable liquid water, water is metastable and can form Ice III or Ice Ih. These critical point parameters pertaining to pressure and temperature define the coldest conditions that water, biological materials, and aqueous substances can remain in liquid state with no possibility of freezing (phase change). According to embodiments, pressure applied to biological and aqueous materials is increased correspondingly with decreased temperature so as to avoid the phase change of liquid water and formation of ice. As described herein, preserving biological material such as cells, tissues, organelles, organoids, molecules, organs, and/or organisms under environmental conditions of elevated pressure (above atmospheric) and temperatures below the freezing temperature of water (i.e., melting temperature) at atmospheric pressure (Earth's surface), supresses enzymatic and overall metabolic activity. As temperature decreases and pressure increases this suppression transitions into cryostasis, a state of suspended animation with virtually no metabolic activity.

Thus, one aspect of the invention relates to storing biological and other aqueous-based materials in a state of suspended animation, i.e. cryostasis. The suspension of metabolism (aerobic and anaerobic), apoptosis and/or necrosis during cryostasis provides for the long-term preservation (i.e., banking) of organic and inorganic aqueous-based materials. The lower the storage temperature, and greater the pressure, the greater the depth of the state of stasis.

Embodiments thus differ from prior approaches that purportedly achieve preservation or storage of biological materials using methods in which reduced pressure is initially applied, and temperature is decreased, with no further reduction in pressure applied as temperature is further decreased. Such prior approaches rely on an observed increase in pressure that occurs upon a further lowering the temperature. The observed increase in pressure in such prior approaches is alleged to prevent the formation of ice (phase change), thereby resulting in storage without damage to the biological material. However, it is suggested herein that the observed increase in pressure can only manifest through the phase change of water in which ice is formed, with deleterious effects on the biological material. In contrast, as discussed above, embodiments described herein avoid the phase change and formation of ice by continuing to increase the pressure applied as temperature is decreased.

In addition, embodiments also differ from prior approaches that rely partially or exclusively on the phase change of water from liquid to ice to generate high pressure in a storage compartment. Such prior approaches do not allow the pressure inside the storage compartment to be controlled, and produce damaging ice inside the storage compartment. In contrast, embodiments described herein use a pressure generator and a drive liquid to pressurize the pressure vessel, allowing precise control of the pressure inside the pressure vessel, and avoid freezing of the drive liquid (which may be water) by applying sufficient pressure to the drive liquid. Embodiments that use water as the drive liquid may conveniently be used to store biological material such as water-dwelling organisms (e.g., fresh water, salt water, etc.) in their natural medium.

Preservation of aqueous-based materials in a non-frozen state can be extended beyond the above described use of pressure to depress the freezing (melting) point to ˜−22° C. There are three means of achieving further freezing point depression (FPD):
1) Supercooling: Aqueous-based materials can be supercooled under pressure where a metastable liquid state can be maintained to at least −92° C.
2) Colligative freezing point depression: Addition of soluble substances to water further depresses the freezing point of the solution below ˜−22° C. under ˜210 MPa. The additional FDP will be equal to 1.86° C. per each mole of colligatively acting solute.
3) Non-colligative freezing point depression: Non-colligative agents provide an additive freezing point depression by means of ice inhibiting or ice binding agents, thus preventing, inhibiting, controlling, and/or sequestering ice crystal growth.

The three methods described above can be used individually or in concert to lower the storage temperature of unfrozen materials below ˜−22° C. under ˜210 MPa. In various embodiments, the storage temperature may be from about 0° C. to about −22° C., from about −5° C. to about −22° C., from about −10° C. to about −22° C., or from about −15° C. to about −22° C. Employing these techniques will extend preservation time for materials requiring cryostasis.

The environmental conditions for storage at or near pressure of ˜210 MPa and temperature of or near ˜−22° C. require a pressure vessel, and a device capable of generating pressure to pressurize and de-pressurize the pressure vessel. A vessel capable of containing these pressures without failing may be made of steel, stainless steel, titanium, or some other appropriate material. The vessel needs to have a way of loading and removing the material stored, and a way of connecting a pressure generator to the vessel. The pressure generator (hydraulic, pneumatic, but not limited to either) can be operated manually, optionally using a timer or controller to control the rate of pressurization and de-pressurization. Alternatively, the pressure generator can be automated and actuated mechanically, pneumatically or hydraulically, or by other means, and controlled by an electrical, electronic, computer or mechanical analog, or other controller. One embodiment includes a hydraulic pressure generator.

A hydraulic pressure generator may be connected to the pressure vessel via a system of pipes, valves, junctions, fittings, pressure gauge(s), etc., that conduct the drive liquid. In such embodiments a drive liquid reservoir may be employed to hold the drive liquid. Examples of drive liquid include, but are not limited to, propylene glycol (PEG), ethylene glycol (EG), oil, petroleum, fish oil, mineral oil, vegetable oil, water, seawater, and any combination thereof.

In order to decrease or increase temperature, the pressure vessel may be operably connected to a controlled cooling and heating device, system, etc., that includes a heat transfer medium. The heat transfer medium can be either fluid or solid. For example, in the case of a cooling/heating system using fluid as the heat transfer vehicle, a container is required to contain the medium, supplied with either a cooler/heater. The heater can be separate from the cooler with its own temperature sensor and temperature controller, or they can be integrated.

A temperature controller may be used to control the cooler/heater based on temperature data provided by a temperature sensor immersed in the heat transfer medium and/or inserted into the pressure vessel. The temperature controller can either be computer software, or a stand-alone controller, microprocessor, or other type of control. The sensor can be a thermocouple, thermistor, RTD (Resistance Thermal Device), or any other appropriate device.

A cooling/heating system using fluid as the heat transfer medium requires a mixing unit, or some other device, to provide constant mixing of the transfer media. Mixing is important for efficient, and better-controlled method of heat transfer, enabling uniform temperature throughout the fluid enclosure, and preventing thermoclines. A pressure gauge, or other measuring/monitoring device, is used to monitor pressure. This can either be, but not limited to, an analog or digital gauge or a pressure transducer connected to a display, or a data acquisition system (DAQ) attached to a computer that displays and records the pressure, a controller, etc. Temperature at or near the interior of the pressure vessel and of the fluid (e.g., air) in the enclosure is monitored with temperature sensors (thermocouples, thermometers, thermistors, RTDs or other suitable device(s)), and data strings may be displayed and/or recorded using a DAQ and computer system, or other system. A thermometer, or other temperature sensor, can be immersed or partially immersed in the fluid in the enclosure to monitor temperature. The pressure vessel remains in the fluid during cooling and warming, and during periods of equilibration.

The cooling/heating and pressure of the system can be integrated and controlled by a single controller utilizing temperature and pressure sensors. Alternatively, the temperature system can be controlled during cooling/warming by a single controller using one or more temperature sensors while the pressure generator operates separately using its own controller and sensor. The cooler/heater and pressure generator can each use their own sensor and controller. One embodiment integrates all three components: heater, cooler, pressure generator into a single control, monitoring, and recording device. The entire high-pressure/low-temperature system controls and monitoring devices may be automated using various control techniques employing diverse equipment and methodologies.

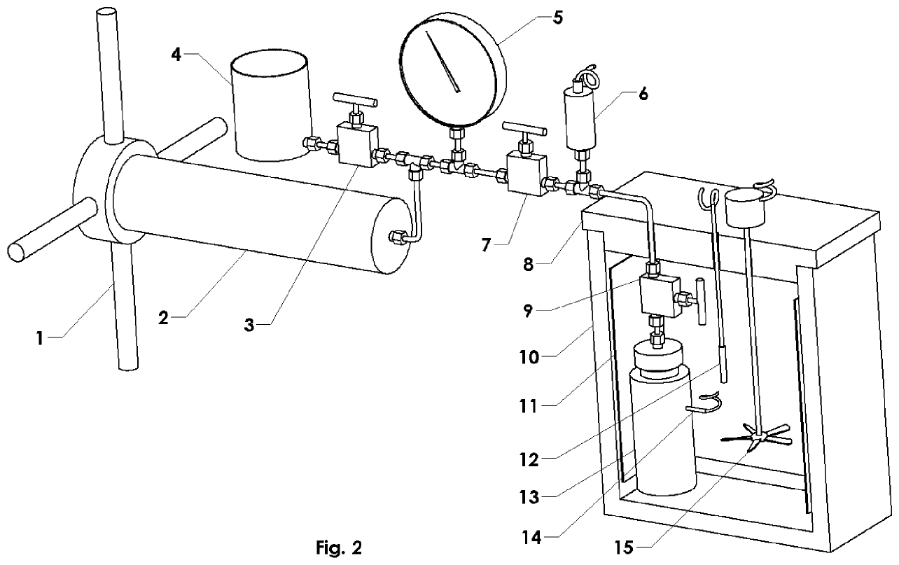

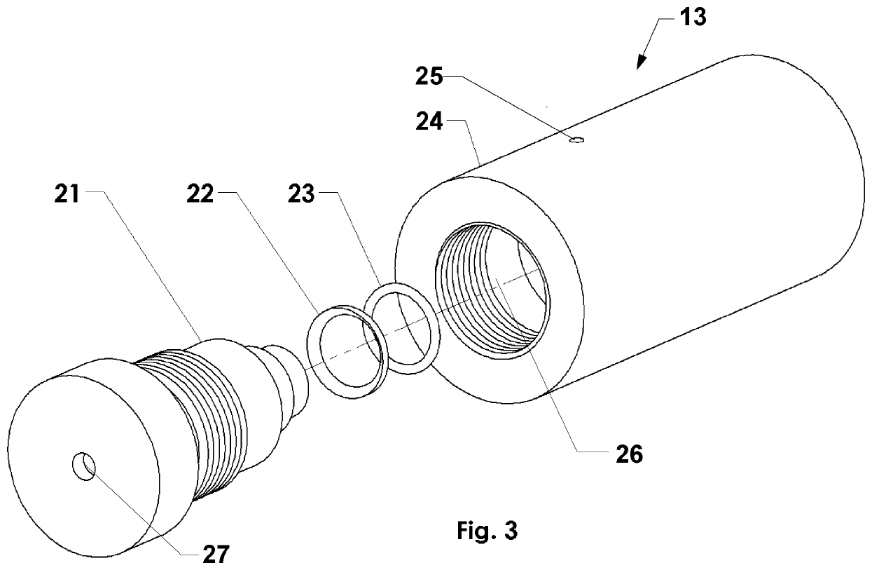

In one embodiment (see FIG. 2), fluid (e.g., air) is used as the heat transfer medium. The apparatus includes a pressure vessel 13 (FIG. 3) capable of containing pressures up to at least 276 MPa without failing; made of steel, stainless steel, titanium, or some other appropriate material, with a removable top 21, and a port 27 for connecting the pressure generator 2 to the pressure vessel 13. The fluid-driven pressure generator 2 can be operated manually, optionally using a separate timer to control the rate of pressurization and de-pressurization. Alternatively, the pressure generator can be actuated mechanically, pneumatically, or hydraulically, etc., and controlled by an electrical, electronic, computer, or mechanical analog controller. For example, the pressure generator may be mechanically driven and computer controlled.

The pressure generator may be connected to the pressure vessel by a system of pipes, valves, junctions, fittings, pressure gauge(s) and hydraulic fluid reservoir (see FIGS. 2 and 3). In order to decrease or increase temperature of the pressure vessel 13, a controlled cooling and heating system may be used. In the case of a cooling/heating system using fluid (e.g., air) as the medium for heat transfer, an insulated container 10 is required to contain the cold/heat sink. A compressor and heat rejection unit can either be housed in the same container outside the cooling/warming device, or they can be in a separate enclosure and connected to the cooling device by insulated pipes.

One embodiment of a mechanical refrigeration system employs a cylindrical reciprocating compressor, optionally with no power surge during start up, and utilizes PID (Proportional-Integral-Derivative) controls. The heater can be separate from the evaporator with its own temperature sensor and temperature controller, or integrated with the evaporator, sharing the same controls. A temperature controller utilizing PID controls the refrigerator/heater based on temperature data provided by a temperature sensor immersed in the heat transfer medium (fluid), or inserted in the pressure vessel. One embodiment uses PID controls for temperature stability and RTD (Resistance Thermal Device) sensors for accuracy and precision.

In one embodiment the refrigeration system, using fluid as the heat transfer medium, has an evaporator as tall as the linear volume of the storage area of the pressure vessel, and a mixer to provide uniform temperature throughout the interior of the storage compartment. In one embodiment the access is from above, by means of a removable insulated top 8, thus creating a cold well. The pressure vessel resides inside the storage compartment during cooling and heating, pressurization and de-pressurization.

A pressure gauge and a pressure transducer may be used to monitor pressure. In one embodiment the pressure transducer was connected to a data acquisition system (DAQ) that was connected to a computer that displayed and recorded the pressure. A thermistor 12 was immersed in the cold well and a second thermistor 14 was inserted into the pressure vessel 13. The data from these temperature sensors was transferred to a computer (via a DAQ as above), where they are displayed and recorded.

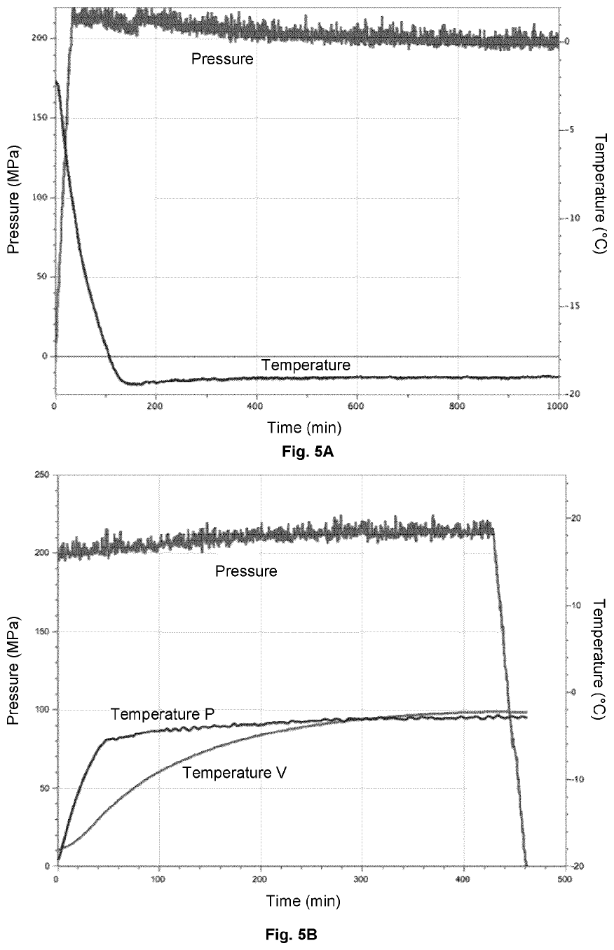

Tissue samples or organs may be obtained immediately post-mortem, perfused, bagged and sealed (see Example 2). Examples of perfusion and storage solutions include UW® Solution (Bridge to Life), CoStorSol®, Celsior®, Custodiol® HTK, Perfadex®, MACS® Tissue Storage Solution (Miltenyi Biotec), FW (Frodin-Wolgast), Sack′, WMo-II, and Lifeport Liver transporter solution. Body heat may be removed by submersing the bagged sample into a solution previously cooled to sub-zero temperature. The tissues and/or organ may then be inserted into the pre-cooled pressure vessel filled with drive liquid, the pressure vessel closed, air removed, and the contents pressurized using the pressure generator and cooled (see Example 3 and FIG. 5). The items may be held in cryostasis for a predetermined period or until needed. Recovery may be accomplished by warming the pressure vessel followed by de-pressurization (see Example 4 and FIG. 6). It will be appreciated that different types and sizes of materials (e.g., solutions, cells, organs, organisms, etc.) to be stored may require different rates of pressure and temperature changes during both initial storing and later recovery, as well as different storage temperatures and pressures. Tables 2 and 3 provide non-limiting examples of rates of pressure and temperature changes during both initial storing and later recovery, as well as different storage temperatures and pressures. For example, FIGS. 5A and 5B are plots showing pressure and temperature curves for placing biological material (porcine renal cortex and medulla) into storage, and recovering the biological material from storage, respectively. In FIG. 5B, “temperature P” refers to the temperature inside the refrigeration device, a refrigerated storage compartment in which the pressure vessel was placed, and “temperature V” refers to the temperature of the pressure vessel.

A laboratory prototype was used to validate the efficacy of the methodology and to determine rates of cooling and warming, pressurization and de-pressurization that are not deleterious to biological material. The benchtop device utilizes a PID controlled refrigeration system for controlled cooling of the vertical walls of an insulated enclosure. The enclosure is open at the top and during operation the top is covered with insulation. The refrigeration system and controller are all housed in the same enclosure. Table 1 catalogs some of the materials that were stored including the storage interval used and post-storage condition.

The laboratory benchtop prototype device can be easily scaled up to accommodate entire organisms, such as humans for interplanetary or interstellar space travel. Some additional equipment may be necessary for the storage of organisms due to the weight of pressure vessels large enough to contain, but not limited to, a kidney, a heart, heart-lung or lung(s), a liver, a pancreas or other human or mammalian organs, either individually or in various combinations. An overhead winch or crane and/or a fork lift, or other weight-handling means, may be needed to move vessels and large, high-stability, walk-in or drive-in refrigerator(s) capable of holding temperatures as low as ˜22° C.

The following working examples further illustrate the invention and are not intended to be limiting in any respect.

### WORKING EXAMPLES

Materials: Bagged and sealed kidney cortex sections in UW® solution (see Example 1), pressure vessel with lid (1″ id, 6″ deep interior well, 15″ on exterior) rated to 276 MPa, pressure generator hand-operated wheel (available from High Pressure Equipment Co. (“HIP”) Erie, Pa., USA) capable of producing 210 MPa of hydraulic pressure, high pressure piping system (available from HIP), valves (available from HIP), gauge (available from HIP), pressure transducer (available from Omega Engineering, Eustache QC, Canada), drive liquid reservoir, propylene glycol and water (1:1) solution, referred to herein as drive liquid, a temperature-ramping Ultrahigh Stability Low Temperature Refrigerated POD 110 VAC (referred to herein as “Work POD”)(refrigerator is available from Engel Coolers, Jupiter, Fla., USA, the Work POD was modified with an Auber Proportional-Integral-Derivative (PID) control (available from Omega Engineering RTD, Eustache QC, Canada) and a resistance thermal device (RTD) sensor, thermistor temperature sensors with data acquisition and recording module (DAQ) (available from Vernier Inc., Beaverton, Oreg., USA), 2-inch closed cell foam insulation sheets sized to cover the Work POD, computer, analog timer (available from GraLab Corporation, Centerville, Ohio, USA), 30 cm Halstead Forceps, lid closing bar, ⅝ inch open end wrench, an isothermal Ultrahigh Stability Low Temperature Refrigerated POD 110 VAC (referred to herein as “Storage POD”) with PID control, operable to −25° C., with a cradle for the pressure vessel. Programming of temperature ramping of the Work POD was done using an instruction manual from Auber Instruments of Alpharetta, Ga., USA entitled SYL-2352P Ramp and Soak PID Temperature Controller, version 1.4 (February 2017). Porcine Kidneys, 300 mM saline (NaCl+H₂O), plastic bags 0.002 in (Mil) thick wall×1 L volume, NaCl 3 M in H₂O 6 L, plastic containers with Lids 3.5 L each, Ultrahigh Stability Refrigerated 12 VDC POD set to −5° C., Ultrahigh Stability Refrigerated POD 110 VAC POD set to −2° C., UW® Solution, Syringe 60 mL with 20 gage Biopsy Needle, Forceps (4), Lancet, Scalpel, Tome Blade, Scales (0.1 gram), Digital Thermometer (resolution 0.1° C.), 6×6 cm 2 Mil low density polyethylene plastic bags (available from International Plastics, Greenville, S.C., USA), heat sealer.

### Example 1. Apparatus for Preservation of Biological Material

Referring to FIG. 2, one embodiment of a pressure-temperature apparatus is shown that includes several components operably connected together via pressure pipes. In FIG. 2, starting from the left side, a drive liquid reservoir 4 that stores drive liquid is connected to a drive liquid isolation valve 3 that is capable of being in an open position that allows drive liquid to flow into the piping or in a or closed position wherein drive liquid is prevented from flowing. At this point in the line, there is a T-junction whereby a pipe joins that leads from a pressure generator 2, which includes an actuator, in this case a hand-operated wheel 1. The pressure generator 2 is operably connected and pressure can be added or removed from the pipe by rotating the hand-operated wheel 1 in an appropriate direction. In other embodiments the actuator may include a motor, servo, or other device that is capable of receiving a control signal (e.g., from a controller such as a microprocessor, computer, etc.) and adjusting the pressure provided by the pressure generator according to the control signal, thereby enabling partially or fully automated control of the pressure. Following this T-junction, the line continues and has a pressure gauge (e.g., which may be digital or analog) 5 that displays a pressure reading. In embodiments with partially or fully automated control the pressure, the pressure gauge includes a pressure transducer that provides a pressure signal to the controller. The next component is a pressure generator isolation valve 7 that allows the pressure-inducing upstream portion of the line to be closed off from the downstream portion at this point. Some embodiments my include a pressure transducer 6 that senses pressure in the line and converts the pressure to a pressure signal, which may be directed to a controller, microprocessor, computer, etc. The line then enters a refrigerated compartment 10, which has an insulated cover 8. The refrigerated compartment 10 may optionally be operably connected to a controller to provide fully or partially automated control of the temperature within the compartment. The line then leads to a pressure vessel isolation valve 9, which allows the pressure vessel 13 to be closed off from the pipe line. The line then connects to the pressure vessel 13 which houses the material to be preserved, as well as drive liquid. Other components in the refrigerated compartment 10 may include a cooler/heater 11 optionally with an interface including, e.g., a digital-to-analogue converter (DAC) so that operation of the heater/cooler 11 may be partially or fully automated using a controller, a temperature sensor for refrigeration control 12, a temperature sensor 14 for monitoring the pressure vessel interior, and a circulating fan or stirrer 15. The temperature sensor for refrigeration control 12 and the temperature sensor 14 for monitoring the pressure vessel interior, which may be implemented with, e.g., thermistors, produce corresponding temperature signals. The temperature signals may be directed to a controller, microprocessor, computer, etc., for monitoring and/or recording the temperatures, and optionally for use in partially or fully automating the apparatus.

Thus, one embodiment includes a controller operably connected to one or more of the temperature sensors, the refrigerated compartment, the pressure transducer (or pressure gauge), the actuator of the pressure generator, and the heater/cooler, so that operation of the apparatus may be partially or fully automated. For example, the controller may control cooling/heating and pressure of the system apparatus. Alternatively, the temperature system can be controlled during cooling/warming by a single controller using one or more temperature sensors while the pressure generator operates separately using its own controller and sensor. One embodiment integrates heating, cooling, and the pressure generator with a single controller that monitors, records, and modulates pressure and temperature.

Referring to FIG. 3, an expanded view of one embodiment of pressure vessel 13 is shown and includes a pressure vessel top 21, a retaining ring 22, an O-ring seal 23, a pressure vessel body 24, an overflow channel and thermistor well 25.

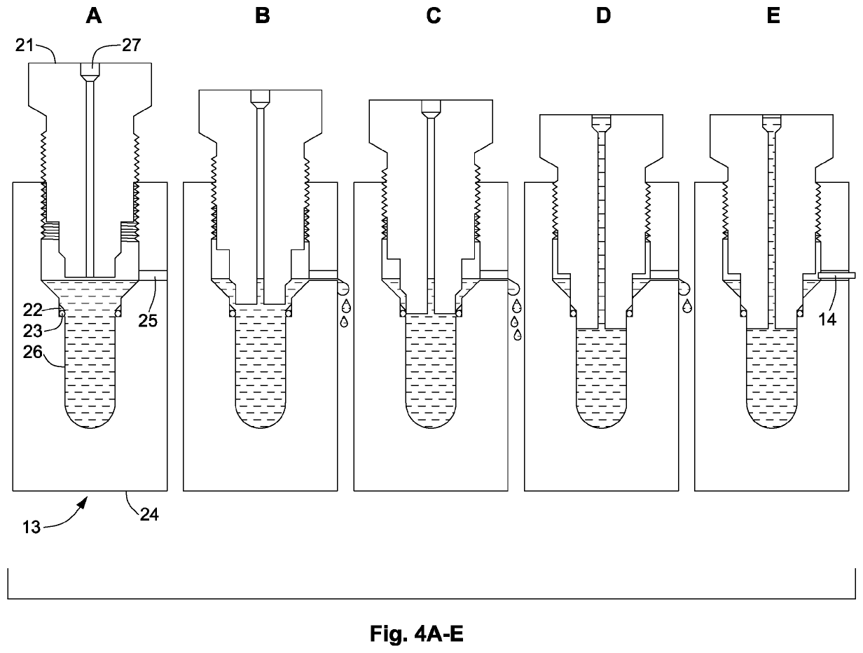

FIGS. 4A-4E sequentially depict assembly of the pressure vessel 13 and pressure vessel top 21 including overflow of drive liquid at the overflow channel and thermistor well 25. Once fully assembled (FIG. 4E), the overflow channel and thermistor well 25 are sealed from the sample well 26 inside the pressure vessel 13, and it houses a thermistor 14 to measure the temperature of the housing. The thermistor 14 is placed in the overflow channel and thermistor well 25 located near the sample well that houses the biological material and drive liquid. Placement closer to the sample well would require a hole near or into the pressurized cavity of the pressure vessel. Such hole could possibly cause failure of the pressure vessel when pressurized. Although the distance separating the thermistor from the sample well may cause the actual temperature of the biological material to lag behind the temperature measured by the thermistor because of poor thermal conductivity of stainless steel, the lag has proven to be acceptable as the cooling rate is low.

FIGS. 5A and 5B are plots showing exemplary pressure and temperature curves for placing biological material (in this case, porcine renal cortex and medulla) into storage, and recovering the biological material from storage, respectively. In FIG. 5B, “temperature P” refers to the temperature inside the refrigeration compartment in which the pressure vessel was placed, and “temperature V” refers to the temperature of the pressure vessel as obtained by a thermistor placed in the overflow channel and thermistor well of the pressure vessel. Of course, different types and sizes of materials (e.g., solutions, cells, organs, organisms, etc.) to be stored may require different rates of pressure and temperature changes during both initial storing and later recovery, as well as different storage temperatures and pressures.

### Example 2. Preparation of Porcine Kidney Cortex Biopsy Sections for Preservation

Obtaining and Preparing Porcine Kidney

Porcine kidneys were obtained from a Canada Food Inspection Agency (CFIA) approved abattoir as soon after post mortem as possible. Inspected kidneys were incised by the CFIA Inspector. Upon receipt, kidneys were separated, rinsed with 300 mM saline, perfused with UW® solution, rinsed with UW® solution, and placed in a 1 L plastic bag and sealed. The bag of kidneys and UW® solution was plunged in to 3 M saline at −5° C. (plunge solution). The plunge solution was housed in a 3.5 L plastic tub located in a 12 VDC POD. Each tub cooled a maximum of three (3) 150 gram kidneys to −1° C. (thermal mass limit for volume and temperature of refrigerant). The tub lid was fitted over the ends of the plastic bags and locked into place. The kidneys remained in the −5° C. plunge solution for 45 minutes to 1 hour. One 6×6 cm 2 Mil plastic bag was marked with a specimen (kidney) number. A 60 cc syringe was fitted with a 20 gage biopsy needle and filled with 50 mL of UW® solution at −1° C. and held in an incubator until needed.

Taking a Section of Kidney

Kidneys were removed from the plunge solution and biopsy sections were prepared individually. A bagged kidney was taken from the plunge solution and the kidney was removed from its bag. The internal temperature of the kidney was determined using a probe on a digital thermometer and the value was recorded. Any residual fat or membrane was removed, and the weight of the kidney was determined and recorded. A longitudinal incision was made, using either a scalpel or tome blade, and a section of cortex was removed. The cortex section was 2-3 cm in length and 1-1.5 cm wide. The cortex section should not contain medulla and should have only one incised face.

Cortex biopsy section removed for preservation 7-10 mL of UW® solution at −1° C. was injected into a 6×6 cm 2 Mil plastic bag. The cortex section was placed into the bag such that the incised face was in contact with the bag wall against a boundary layer of UW® solution. Additional UW® solution at −1° C. was injected into the bag, as needed, to cover the cortex section. The bag was closed and compressed removing all air. The bag was sealed with a heat sealer and excess plastic was trimmed off. The bag was placed into a refrigerated POD at −2° C. until all of the cortex sections for storage were prepared.

### Example 3. Storage Process for Storing Biological Material at −18° C. and 193 MPa

Setup

The following steps were performed one day prior to storage of biological samples, using an apparatus based on that shown in FIG. 2 and described in Example 1. A Storage POD was set to and maintained at −18° C. The empty pressure vessel 13 (see FIG. 2) was placed into the Work POD 10. The Work POD's temperature controller was set to −2° C. and the interior temperature of the pressure vessel was ramped to −2° C. over 6 hours. Once at −2° C., the pressure vessel 13 was allowed to stabilize for 8 hours. The Auber PID was programmed. The pressure vessel 13 was connected to the piping system. Two layers of 2-inch closed foam insulation were placed on top of the Work POD. A first thermistor 14 was inserted into overflow channel/thermistor well 25 (see FIG. 3) in the pressure vessel 13. A second thermistor 12 was positioned next to the pressure vessel 13 in the interior of the Work POD. The computer was powered on, connected to the data acquisition system (DAQ), and Vernier Lab View Software was configured to record readings every 10 seconds for 5,000 minutes and recording was started.

Samples Placed in Work POD, Ramped to −18° C. and 193 MPa, and Stabilized

The following steps were performed on the day of sample collection. Porcine Kidney Cortex Biopsy Samples were prepared and held at −2° C. as described in Example 2. Two closed foam insulation sheets were removed from the top of the Work POD. The first thermistor 14 was removed from 25 and the pressure vessel 13 was detached from the piping system. The pressure vessel 13 was removed from the Work POD and positioned on its stand from the Work POD. The pressure vessel lid 21 was unthreaded and removed. Halstead Forceps (0 cm) were used to place a first set of two (2) bagged and sealed samples side-by-side into the sample well 26 of the pressure vessel, then a second set of two (2) bagged and sealed samples were placed above the first set. Attention was required to leave enough room so that the lid 21 fit into the pressure vessel 13 without contacting the samples. Drive liquid entered the pressure vessel and while lid 21 was closed by threading it into the pressure vessel 13 with the pressure vessel isolation valve 9 open, excess drive liquid discharged through overflow channel/thermistor well 25 in the side of the pressure vessel 13 (see FIGS. 4A-4E). The overflow channel and thermistor well 25 was observed until drive liquid ran freely out of it with no air bubbles. Once lid 21 had blocked the overflow channel, drive liquid was discharged from the top of pressure vessel isolation valve 9 until no air bubbles were observed. Lid 21 was tightened onto the pressure vessel 13 until snug using a strap wrench and a lid closing bar. The pressure vessel 13 was transferred into the Work POD and connected to the piping system. The fitting that connects the pressure vessel to the piping system was finger tightened. The Drive liquid Isolation Valve 3, located up-stream from the pressure generator 2, was opened. The pressure generator isolation valve 7 located downstream from the pressure generator 2 was also opened. A fitting collar on the pipe fitting that connects the piping system to the pressure vessel 13 was checked and tightened. The pipe fitting was inserted into the pressure vessel 13 and tightened by turning the threads one turn. The fitting connecting the pressure vessel isolation valve 9 to the piping system (40 ft/lbs) was tightened until it was snug. The drive liquid reservoir isolation valve 3 was closed, and a check was performed to ensure that the pressure generator isolation valve 7 and the pressure vessel isolation valve 9 were open one full turn. The first thermistor 14 was replaced in the overflow channel and thermistor well 25.

Pressure was increased inside the pressure vessel, and the temperature was programmed to decrease gradually (see, e.g., Table 2). The controller on the Work POD was programmed to ramp from −2° C. to −18° C. at one rate. The tissue cools much more slowly than the Work POD because of the coefficient of heat transfer across the pressure vessel material (e.g., stainless steel). The count-down Gra-Lab timer was set to 20 minutes, and was used to control the rate of pressurization. Using the pressure generator 1, the pressure vessel 13 was pressurized at a rate of 1,000 psig/minute (6.9 MPa) in increments of 200 psig (1.4 MPa) every 12 seconds to 30,000 psig (210 MPa). The system was allowed to ramp and soak for 12 hours. It was noted that cooling resulted in a loss of 2,000 psig (13.8 MPa). The temperature and pressure were allowed to stabilize for 12 hours. At that time, the pressure was adjusted to 28,000 psig (193 MPa) and the system was allowed to stabilize for an additional 6 hours.

Transfer from Work POD to Storage POD, Stored at −18° C. and 193 MPa

Once the pressure vessel was stabilized at −18° C. and 193 MPa in the Work POD, it was ready to be transferred for storage in the Storage POD, which was isothermal at −18° C. The pressure vessel isolation valve 9 was closed. The drive liquid reservoir isolation valve 3 was opened, dropping the pressure in the piping system and pressure generator to ambient. Using a ⅝″ open end wrench, the pipe fitting from the pressure vessel isolation valve 9 was detached. The drive liquid reservoir isolation valve 3 was closed. Recording of temperature and pressure was stopped and data was saved on the computer. The temperature sensor 14 was removed from the pressure vessel 13. The top of the Storage POD was opened. The pressure vessel 13 was lifted out of the Work POD and transferred into a cradle inside the Storage POD, which was isothermal at −18° C. The top of the Storage POD was closed. The samples in the pressure vessel were allowed to soak for 10 days at −18° C. (Note: storage interval can vary).

### Example 4. Recovery of Biological Material from Storage POD at −18° C. and 193 MPa to Ambient Temperature and Pressure

Samples were located in the Storage POD −18° C. and 193 MPa. When recovery of a stored sample was desired, the following steps were followed.

The following steps were conducted 6 hours before recovery. The Work POD was started and the controls were set to bring the Work POD temperature to −18° C. It was ensured that both layers of 2″ thick closed foam insulation were located on top of the Work POD. The computer was started and a program (e.g., Graphical Analysis™ 4, available from Vernier, Beaverton, Oreg., USA) was launched for recording temperature and pressure (e.g., 1 sample/10 seconds).

Six hours after the above steps, the following recovery protocol was performed. It was confirmed from the temperature data record that the Work POD had been isothermal at −18° C. for more than 4 hours. The pressure generator isolation valve 7 was opened. The drive liquid reservoir isolation valve 3 was closed. The cover of the Storage POD was opened and the pressure vessel assembly was removed and transferred to the Work POD. The base of the pressure vessel was placed into its stand at the bottom of the Work POD. The piping system was connected to the pressure vessel isolation valve 9 and the fitting was turned 1 turn. The drive liquid reservoir isolation valve 3 was opened. A bleed hole in the pressure vessel isolation valve was observed until no air bubbles had appeared for 15 seconds. The fitting connecting the pressure vessel isolation valve 9 to the piping system was tightened firmly. The drive liquid reservoir isolation valve 3 was closed. Using the pressure generator 1, the piping system was pressurized to 193 MPa (28,000 psig). Heat of compression was allowed to dissipate for 10 minutes. Pressure was adjusted to 193 MPa (28,000 psig). The pressure vessel isolation valve 9 was opened. A drop in system pressure was avoided since a reduction in pressure below 171.1 MPa (24,908 psig) can result in freezing and loss of specimen viability.

The Work POD was ramped from ˜18° C. to −2° C. at a rate of 0.05° C./min (3.0° C./hour, 5.5 hr total for 16° C. ΔT; during the warming, it was observed that internal pressure increased to 209 MPa (about 30,000 psig). The Work POD was soaked for 1 hour (minimum). Using the pressure generator hand-operated wheel 1, the pressure vessel was de-pressurized by 1,000 psig/minute (6.9 MPa) in increments of 200 psig (1.4 MPa) every 12 seconds for 30 minutes until ambient pressure was reached.

The pressure vessel 13 was disconnected from the piping system by loosening the fitting to the pressure vessel isolation valve 9. The pressure vessel 13 at −2° C. was opened by un-threading its top 21 and the top was removed from the vessel. Each of the four samples was removed from the vessel interior using 30 cm hemostats.

The samples were stained using DAPI/PI (see Table 1 for full names) and analyzed for viability. Results are shown in Table 1. Unused parts of the stored kidney biopsy sections were frozen for subsequent Caspase/Adenosine Triphosphate (ATP) analyses.

TABLE 1
Materials preserved unfrozen using sub-zero ° C. storage at elevated pressure.

| | Storage | Storage | | | | |
|---|---|---|---|---|---|---|
| Material | Duration | Temperature | | Condition | | |
| stored (N) | (Solution) | and Pressure | | after storage | Analysis used | Notes |
| Water | | −20° | C. | unfrozen | Ice nucleation | Theoretically water can be |
| (N = 33) | | 30,000 | psi | | | stored unfrozen indefinitely |
| | | 207 | MPa | | | at −20° C. & 30,000 psi |
| Porcine kidney | 2 weeks | −18° | C. | Mean cell | PI/DAPI | No increase in cell |
| biopsies | (UW) | 30,000 | psi | viability = | Caspase | apoptosis post-storage |
| (N = 48) | | 207 | MPa | 94 ± 3% S.D. | Visual | No necrosis or deterioration |
| Porcine kidney | 4 weeks | −18° | C. | Mean cell | I/DAPI | No necrosis or deterioration, |
| biopsies | (UW) | 30,000 | psi | viability = | visual | No discoloration post-storage |
| (N = 12) | | 207 | MPa | 94 ± 3% S.D. | | |
| Porcine heart | 8 days | −8° | C. | 100% cell | PI/FDA | Organ not viable when |
| (N = 1) | (phosphate | 18,000 | psi | mortality | Visual | received, cut open |
| | buffer) | 124 | MPa | | | |
| Rabbit heart | 6 weeks | −18° | C. | ~90% cell | PI/FDA/DAPI | No necrosis or deterioration, |
| (N = 3) | (phosphate | 30,000 | psi | viability | visual | No discoloration post-storage |
| | buffer) | 207 | MPa | | | |
| Rabbit heart | 7 days | −18° | C. | ~90% cell | PI/FDA/DAPI | No necrosis or deterioration, |
| (N = 16) | (phosphate | 30,000 | psi | viability | visual | No discoloration post-storage |
| | buffer) | 207 | MPa | | | |
| Rabbit kidney | 10 days | −18° | C. | ~90% cell | PI/FDA/DAPI | No necrosis or deterioration, |
| (N = 12) | (CryoStasis) | 30,000 | psi | viability | visual | No discoloration post-storage |
| | | 207 | MPa | | | |
| Rat heart | 7 days | −18° | C. | ~90% cell | PI/FDA | Good condition |
| (N = 2) | (CryoStasis) | 30,000 | psi | viability | Visual | No necrosis |
| | | 207 | MPa | | Langendorf | No discoloration |
| Rat kidney | 8 days | −20° | C. | ~90% cell | PI/FDA | Organs intact, unchanged |
| (N = 4) | (CryoStasis) | 30,000 | psi | viability | Visual | compared to before storage |
| | | 207 | MPa | | Morphology | |
| Bovine | 7 days | −18° | C. | 30% cell | PI/FDA/DAPI | Air bubbles problem |
| spermatozoa | (Tolga) | 30,000 | psi | viability | MTT assay | |
| (N = 3 × | | 207 | MPa | | motility | |
| 300,000) | | | | | | |
| Oyster larvae | 23 days | −18° | C. | 5-20% | PI/FDA | Survival was age-dependent; |
| (N = 6 cohorts) | (sea water) | 30,000 | psi | viability | Morphological | best in 3-week veliger larvae |
| | | 207 | MPa | | Motility | |
| Mud minnows | 7 days | −18° | C. | None | Visual | Fish intact, no damage |
| (N = 7) | (water) | 30,000 | psi | survived but | Morphological | Could not function when |
| | | 207 | MPa | unchanged | Physiological | returned to aquarium |
| HEK293 cells | 12 hours | 20° | C. | 5% cell | PI/FDA | Poor survival at 20° C. |
| (N = 3 × | (DMEM) | 15,000 | psi | viability | MTT assay | and high pressure |
| 100,000) | | 104 | MPa | | | |
| Mitochondria | 1 day- | −18° | C. | Always intact | DAPI | Intact in all studies |
| (various | 1 month | 30,000 | psi | Function | MTT assay | Function confirmed in some |
| sources) | (various | 207 | MPa | confirmed in | | studies |
| | media) | | | some trials | | |
| Catalase | 3 days | −18° | C. | Function | H₂O₂ | Test of high pressure |
| (N = 2) | (water) | 30,000 | psi | confirmed | | effect on enzymes |
| | | 207 | MPa | | | |
| PEG | 1 day- | −18° | C. | No change | Osmometry | FPD unchanged |
| (N = 100+) | 1 month | 30,000 | psi | | | |
| | | 207 | MPa | | | |
| EG | 1 day- | −18° | C. | No change | Osmometry | FPD unchanged |
| (N = 12) | 1 month | 30,000 | psi | | | |
| | | 207 | MPa | | | |
| Phosphate | 1 day- | −18° | C. | No change | Cell survival | Cells survived |
| buffer | 1 month | 30,000 | psi | | | |
| (N = 16) | | 207 | MPa | | | |
| DMEM | 12 hours | 20° | C. | No change | Cell survival | Cells survived |
| (N = 3) | (DMEM) | 15,000 | psi | | | |
| | | 104 | MPa | | | |
| U. of Wisconsin | 1 day- | −18° | C. | No change | Cell survival | Cells survived |
| Solution (UW ®) | 1 month | 30,000 | psi | | | |
| (N = 60) | | 207 | MPa | | | |
| CryoStasis | 1 day- | −18° | C. | No change | Osmometry | FPD unchanged |
| Solution | 1 month | 30,000 | psi | | | AFP activity unchanged |
| (N = 22) | | 207 | MPa | | | |
| Antifreeze | 3 days | −18° | C. | No change | Osmometry | FPD unchanged |
| Proteins | (CryoStasis) | 30,000 | psi | | | |
| (N = 4) | | 207 | MPa | | | |
| Bacteria & | 23 days | −18° | C. | Cells intact | Microscopic | Cells intact & motile |
| algae | (sea water) | 30,000 | psi | & motile | | |
| (N = 6) | | 207 | MPa | | | |
| Sea water | 23 days | −18° | C. | No change | Osmometry | FPD unchanged |
| (N = 6) | | 30,000 | psi | | Larval | |
| | | 207 | MPa | | survival | |

Acronyms: PI (Propidium Iodide); FDA (Fluorescein Diacetate); DAPI (4′,6-diamidino-2-phenylindole); DMEM (Dulbecco Modified Eagle Medium); PEG (Propylene Glycol); EG (Ethylene Glycol); FPD (Freezing Point Depression). “CryoStasis” and “Tolga” refer to aqueous based solutions. UW ® (Southard, J. H. et al.,, Transplantation Reviews 7(4): 176-190, 1993).

TABLE 2
Temperature changes and corresponding
pressures for various materials.

| Material Stored | Pressure protocol (psi) | | Temperature Protocol (° C.) | |
|---|---|---|---|---|
| Water | 1) | 0 to 5,000; | 1) | 0° C. to −2.5° C.; |
| (N = 33) | | 5,000 to 10,000; | | −2.5° C. to −6.0 C. °; |
| | | 10,000 to 15,000; | | −6.0° C. to −9.2° C.; |
| | | 15,000 to 20,000; | | −9.2° C. to −12.5° C.; |
| | | 20,000 to 25,000; | | −16.5° C. to −20.0° C. |
| | | 25,000 to 30,000 | | |
| | 2) | 0 to 10,000; | 2) | 0° C. to −6.0° C.; |
| | | 10,000 to 20,000; | | −6.0° C. to 12.5° C.; |
| | | 20,000 to 30,000 | | −12.5° C. to −20.0° C. |
| | | reversed for warming | | |
| | | all soaks 6 hours | | |
| | | Ramp rates: | | |
| | | 5,000 psi/min | | |
| | | 3,000 psi/min | | |
| | | 1,000 psi/min | | |
| | | 500/psi/min | | |

| | | |
|---|---|---|
| Porcine kidney | 0 to 30,000, at −18° C. | −2° C. to −18° C. |
| biopsies | pressure has dropped to | 28,000 psi = −19.3° C. |
| (N = 48) | 27,800 psi; reset to 28,000 | |
| | for storage. | |
| | reversed for warming, | |
| | soak 12 hours | |
| | ramp rate: 1,000 psi/min | |
| Porcine kidney | 0 to 30,000, at −18° C. | −2° C. to −18° C. |
| biopsies | pressure has dropped to | 28,000 psi = −19.3° C. |
| (N = 12) | 27,800 psi; reset to 28,000 | |
| | for storage | |
| | reversed for warming, | |
| | soak 12 hours | |
| | ramp rate: 1,000 psi/min; | |
| Porcine heart | 0 to 15,000; | −2° C. to −9.0° C.; |
| (N = 1) | 15,000 to 30,000 | −9.0° C. to −20° C. |
| | reversed for warming, | |
| | soak 4 hours, | |
| | ramp rate: 500 psi/min | |
| Rabbit heart | 0 to 5,000; | 0° C. to −2.5° C.; |
| (N = 3) | 5,000 to 10,000; | −2.5° C. to −6.0 C. °; |
| | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | reversed for warming, | |
| | all soaks 6 hours | |
| | ramp rate 250 psi/min | |
| Rabbit heart | 0 to 5,000; | 0° C. to −2.5° C.; |
| (N = 16) | 5,000 to 10,000; | −2.5° C. to −6.0 C. °; |
| | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | reversed for warming, | |
| | all soak 6 hours; | |
| | ramp rate 250 psi/min | |
| Rabbit kidney | 0 to 5,000; | 0° C. to −2.5° C.; |
| (N = 12) | 5,000 to 10,000; | −2.5° C. to −6.0 C. °; |
| | 10,000 to 15,000; | −6.0° C. to −9.2° C.; |
| | 15,000 20,000; | −9.2° C. to −12.5° C.; |
| | 20,000 to 25,000; | −12.5° C. to −16.5° C.; |
| | 25,000 to 30,000 | −16.5° C. to −20.0° C. |
| | reversed for warming, | |
| | all soak 6 hours; | |
| | ramp rate 1,000/min | |
| Rat heart | 0 to 15,000; | 2° C. to −9.0° C.; |
| (N = 2) | 15,000 to 30,000 | −9.0° C. to −20° C. |
| | ramp rate 500 psi/min | |
| | reversed for warming, | |
| | soak 4 hours | |
| Rat kidney | 0 to 15,000; | 2° C. to −9.0° C.; |
| (N = 4) | 15,000 to 30,000 | −9.0° C. to −18° C. |
| | reversed for warming, | |
| | soak 4 hours | |
| | ramp rate 500 psi/min | |
| Bovine | 0 to 5,000; | 0° C. to −2.5° C.; |
| spermatozoa | 5,000 to 10,000; | −2.5° C. to −6.0 C. °; |
| (N = 3 × | 10,000 to 15,000; | −6.0° C. to −9.2° C.; |
| 300,000) | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | reversed for warming; | |
| | 200 psi/min, | |
| | 2 hour soaks, Tolga | |
| | Tris unfrozen bovine | |
| | extender | |
| Oyster larvae | 0 to 5,000; | 0° C. to −2.5° C.; |
| (N = 6 cohorts) | 5,000 to 10,000; | −2.5° C. to −6.0 C. °; |
| | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | reversed for warming; | |
| | 100 psi/min, | |
| | 2 hour soaks, sea water | |
| Mud minnows | 0 to 5,000; | 0° C. to −2.5° C.; |
| (N = 7) | 5,000 to 10,000; | −2.5° C. to −6.0 C. °; |
| | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | reversed for warming; | |
| | 100 psi/min, | |
| | 2 hour soaks, pond water | |
| HEK293 cells | 0 to 5,000; | 0° C. to −2.5° C.; |
| (N = 3 × | 5,000 to 10,000; | −2.5° C. to −6.0 C. °; |
| 100,000) | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | reversed for warming; | |
| | 200 psi/min, | |
| | 2 hour soaks, DMEM | |
| Mitochondria | Various protocols | Various protocols |
| (various | | |
| sources) | | |
| Catalase | 0 to 30,000 psi | −2° C. to −20° C. |
| (N = 2) | reversed for warming; | |
| | 15,000/min reacted | |
| | with H2O2 after recovery | |

TABLE 3
Temperature changes and corresponding
pressures for various solutions.

| Material Stored | Pressure protocol (psi) | Temperature Protocol (° C.) |
|---|---|---|
| PEG | 0 to 5,000; | 0° C. to −2.5° C.; |
| (N = 100+) | 5,000 to 10,000; | −2.5° C. to −6.0 C. °; |
| | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | 0 to 10,000; | 0° C. to −6.0° C.; |
| | 10,000 to 20,000; | −6.0° C. to −12.5° C.; |
| | 20,000 to 30,000 | −12.5° C. to −20.0° C. |
| | reversed for warming | |
| | all soaks 6 hours | |
| | Ramp rates: | |
| | 5,000 psi/min | |
| | 3,000 psi/min | |
| | 1,000 psi/min | |
| | 500/psi/min | |
| EG | 0 to 5,000; | 0° C. to −2.5° C.; |
| (N = 12) | 5,000 to 10,000; | −2.5° C. to −6.0 C. °; |
| | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | 0 to 10,000; | 0° C. to −6.0° C.; |
| | 10,000 to 20,000; | −6.0° C. to −12.5° C.; |
| | 20,000 to 30,000 | −12.5° C. to −20.0° C. |
| | reversed for warming | |
| | all soaks 6 hours | |
| | Ramp rates: | |
| | 5,000 psi/min | |
| | 3,000 psi/min | |
| | 1,000 psi/min | |
| | 500/psi/min | |
| Phosphate | 0 to 5,000; | 0° C. to −2.5° C.; |
| buffer | 5,000 to 10,000; | −2.5° C. to −6.0 C. °; |
| (N = 16) | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | 0 to 10,000; | 0° C. to −6.0° C.; |
| | 10,000 to 20,000; | −6.0° C. to −12.5° C.; |
| | 20,000 to 30,000 | −12.5° C. to −20.0° C. |
| | reversed for warming | |
| | all soaks 6 hours | |
| | Ramp rates: | |
| | 5,000 psi/min | |
| | 3,000 psi/min | |
| | 1,000 psi/min | |
| | 500/psi/min | |
| DMEM | 0 to 5,000; | 0° C. to −2.5° C.; |
| (N = 3) | 5,000 to 10,000; | −2.5° C. to −6.0 C. °; |
| | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | 0 to 10,000; | 0° C. to −6.0° C.; |
| | 10,000 to 20,000; | −6.0° C. to −12.5° C.; |
| | 20,000 to 30,000 | −12.5° C. to −20.0° C. |
| | reversed for warming | |
| | all soaks 6 hours | |
| | Ramp rates: | |
| | 5,000 psi/min | |
| | 3,000 psi/min | |
| | 1,000 psi/min | |
| | 500/psi/min | |
| University of | 0 to 5,000; | 0° C. to −2.5° C.; |
| Wisconsin | 5,000 to 10,000; | −2.5° C. to −6.0 C. °; |
| Solution | 10,000 to 15,000; | −6.0° C. to −9.2° C.; |
| (N = 60) | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | 0 to 10,000; | 0° C. to −6.0° C.; |
| | 10,000 to 20,000; | −6.0° C. to −12.5° C.; |
| | 20,000 to 30,000 | −12.5° C. to −20.0° C. |
| | reversed for warming | |
| | all soaks 6 hours | |
| | Ramp rates: | |
| | 5,000 psi/min | |
| | 3,000 psi/min | |
| | 1,000 psi/min | |
| | 500/psi/min | |
| CryoStasis | 0 to 5,000; | 0° C. to −2.5° C.; |
| Solution | 5,000 to 10,000; | −2.5° C. to −6.0 C. °; |
| (N = 22) | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | 0 to 10,000; | 0° C. to −6.0° C.; |
| | 10,000 to 20,000; | −6.0° C. to −12.5° C.; |
| | 20,000 to 30,000 | −12.5° C. to −20.0° C. |
| | reversed for warming | |
| | all soaks 6 hours | |
| | Ramp rates: | |
| | 5,000 psi/min | |
| | 3,000 psi/min | |
| | 1,000 psi/min | |
| | 500/psi/min | |
| Antifreeze | 0 to 5,000; | 0° C. to −2.5° C.; |
| Proteins | 5,000 to 10,000; | −2.5° C. to −6.0 C. °; |
| (N = 4) | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | 0 to 10,000; | 0° C. to −6.0° C.; |
| | 10,000 to 20,000; | −6.0° C. to −12.5° C.; |
| | 20,000 to 30,000 | −12.5° C. to −20.0° C. |
| | reversed for warming | |
| | all soaks 6 hours | |
| | Ramp rates: | |
| | 5,000 psi/min | |
| | 3,000 psi/min | |
| | 1,000 psi/min | |
| | 500/psi/min | |
| Bacteria & | 0 to 5,000; | 0° C. to −2.5° C.; |
| algae | 5,000 to 10,000; | −2.5° C. to −6.0 C. °; |
| (N = 6) | 10,000 to 15,000; | −6.0° C. to −9.2° C.; |
| | 15,000 to 20,000; | −9.2° C. to −12.5° C.; |
| | 20,000 to 25,000; | −12.5° C. to −16.5° C.; |
| | 25,000 to 30,000 | −16.5° C. to −20.0° C. |
| | 0 to 10,000; | 0° C. to −6.0° C.; |
| | 10,000 to 20,000; | −6.0° C. to −12.5° C.; |
| | 20,000 to 30,000 | −12.5° C. to −20.0° C. |
| | reversed for warming | |
| | all soaks 6 hours | |
| | Ramp rates: | |
| | 5,000 psi/min | |
| | 3,000 psi/min | |
| | 1,000 psi/min | |
| | 500/psi/min | |
| Sea water | 0 to 5,000; | 0° C. to −2.5° C.; |
| (N = 6) | 5,000 to 10,000; | −2.5° C. to −6.0 C. °; |
| | 10,000 to 15,000; | −6.0° C. to −9.2° C.; |
| | 15,000 20,000; | −9.2° C. to −12.5° C.; |
| | 20,000 to 25,000; | 12.5° C. to −16.5° C.; |
| | 25,000 to 30,000 | −16.5° C. to −20.0° C. |
| | 0 to 10,000; | 0° C. to −6.0° C.; |
| | 10,000 to 20,000; | −6.0° C. to −12.5° C.; |
| | 20,000 to 30,000 | −12.5° C. to −20.0° C. |
| | reversed for warming | |
| | all soaks 6 hours | |
| | Ramp rates: | |
| | 5,000 psi/min | |
| | 3,000 psi/min | |
| | 1,000 psi/min | |
| | 500/psi/min | |

### INCORPORATION BY REFERENCE

The contents of all cited publications are incorporated herein by reference in their entirety.

### EQUIVALENTS

It will be understood by those skilled in the art that this description is made with reference to certain embodiments and that it is possible to make other embodiments employing the principles of the invention which fall within its spirit and scope.

We claim:

1. A method for storing biological material, comprising:
disposing the biological material in a pressure vessel;
filling the pressure vessel with a drive liquid;
displacing air from the pressure vessel and sealing the pressure vessel;
increasing pressure on the drive liquid using a pressure generator and decreasing temperature below 0° C. inside the pressure vessel;
wherein at a selected temperature a selected pressure is applied to the drive liquid using the pressure generator whereby the drive liquid in the pressure vessel is maintained in a stable, liquid state;
wherein freezing of the biological material is prevented at a storage temperature below 0° C. by applying a selected pressure to the drive liquid.

2. The method of claim 1, further comprising
disposing the biological material in a sample bag with a preservation solution;
evacuating air from the sample bag; and
sealing the sample bag;
wherein the preservation solution and the drive liquid are maintained in a stable, liquid state.

3. The method of claim 1 or 2, wherein decreasing the temperature and increasing the pressure comprises increasing pressure from ambient conditions at 1,000 psig/minute (6.9 MPa) in increments of 200 psig (1.4 MPa) to about 30,000 psig (210 MPa), and decreasing temperature from ambient conditions to about −22° C.

4. The method of any one of claims 1 to 3, wherein the biological material comprises one or more of organic molecules, molecular complexes, nucleic acids, saccharides, amino acids, peptides, proteins, enzymes, organelles, organoids, cells, tissues, organs, organisms, and an aqueous solution.

5. The method of any one of claims 2 to 4, wherein the preservation solution comprises water and one or more of biological material, soluble molecules, organic and/or inorganic compounds, material in aqueous suspension, aqueous solution, aqueous mixture, aqueous colloids, aqueous-based material, and material of biological origin.

6. The method of any one of claims 1 to 5, wherein the biological material comprises cells, tissues, organs, or entire organisms.

7. The method of any one of claims 1 to 6, wherein the storage temperature is about −22° C.

8. The method of any one of claims 1 to 7, wherein at the storage temperature the applied pressure is about 30,000 psi (210 MPa).

9. The method of any one of claims 1 to 8, wherein the storage temperature and applied pressure prevent freezing and cell damage by maintaining cells a metastable supercooled liquid state.

10. The method of any one of claims 2 to 9, wherein the preservation solution comprises a solute.

11. The method of claim 10, wherein the solute comprises one or more of antifreeze protein, ice binding protein, antifreeze saccharide, ice binding saccharide, ice binding peptide, and other non-colligative agents.

12. The method of claim 10 or 11, wherein the solute prevents, inhibits, controls, or sequesters ice crystal growth, and/or prevents nucleation of ice.

13. The method of any one of claims 1 to 12, wherein the drive liquid comprises propylene glycol or ethylene glycol, oil, petroleum, fish oil, mineral oil, vegetable oil, water, seawater, any combination thereof.

14. The method of any one of claims 1 to 13, wherein the selected storage temperature is from about −5° C. to about −22° C.

15. Apparatus for storing biological material, comprising:
a reservoir for housing a drive liquid;
a pressure vessel having an internal well adapted for receiving the biological material, the pressure vessel operably connected to the reservoir to receive drive liquid from the reservoir;
a pressure generator operably connected to the pressure vessel and the reservoir, that applies pressure on the drive liquid;
a pressure transducer that provides an indication of the pressure of the drive liquid in the pressure vessel;
a temperature sensor that senses temperature of the pressure vessel; and
a refrigeration device adapted to provide a controlled pressure vessel internal temperature below about 0° C.;
wherein at a selected pressure vessel temperature below about 0° C. the pressure generator applies a selected pressure to the drive liquid to maintain the drive liquid in the pressure vessel in a stable, liquid state.

16. The apparatus of claim 15, further comprising a data acquisition system (DAQ) that acquires data from one or more of the pressure transducer, the temperature sensor, the pressure generator, and the refrigeration device.

17. The apparatus of claim 15 or 16, further comprising a controller operably connected to one or more of the pressure transducer, the temperature sensor, the pressure generator, and the refrigeration device;
wherein the controller monitors and maintains at least one of a selected internal pressure vessel temperature and a selected pressure on the drive liquid in the pressure vessel.

18. The apparatus of any one of claims 15 to 17, further comprising a pressure gauge.

19. The apparatus of claim 17, wherein the pressure generator is automated and driven mechanically, electrically, pneumatically, or hydraulically by the controller.

20. The apparatus of any one of claims 15 to 19, wherein the refrigeration device further comprises a heater.

21. The apparatus of claim 20, wherein the heater comprises a temperature sensor and temperature controller.

22. The apparatus of any one of claims 15 to 21, wherein the refrigeration device comprises proportional-integral-derivative (PID) control.

23. The apparatus of any one of claims 15 to 22, further comprising an evaporator.

24. The apparatus of any one of claims 15 to 23, further comprising at least one valve that, when closed, allows isolation and removal of the pressure vessel from the apparatus;
wherein the pressure vessel retains the applied pressure of the drive liquid when removed from the apparatus.

25. The apparatus of any one of claims 15 to 24, wherein the pressure vessel is made from a material selected from steel, stainless steel, and titanium.

26. The apparatus of any one of claims 15 to 25, wherein the pressure vessel is adapted to withstand an internal pressure of at least about 30,000 psig (210 MPa).

27. A pressure vessel for storing biological material, comprising:
a housing having a cavity including a first portion, and a sample well that receives the biological material and a drive liquid;
the first portion of the housing including an overflow channel that is open to an exterior of the housing;
a lid including a first portion adapted to engage the first portion of the housing whereby a position of the lid within the housing is adjustable over a range from a first position to a closed position;
the lid including a second portion adapted to partially fit into the sample well of the housing;
the first portion of the lid including a port adapted to interface with external equipment;
the lid including a drive liquid channel adapted to conduct drive liquid through the lid between the port and the sample well;
wherein adjusting the lid to the closed position expels excess drive liquid from the sample well via the port and the overflow channel, and the second portion of the lid seals the sample well;
wherein the pressure vessel is adapted to sustain an internal pressure of drive liquid in the sample well of at least about 30,000 psi (210 MPa).

28. The pressure vessel of claim 27, wherein pressure is applied to drive liquid in the sample well by the external equipment via the port.

29. The pressure vessel of claim 28, further comprising at least one valve disposed between the port and the external equipment;
wherein, when closed, the at least one valve isolates the pressure vessel from the external equipment and maintains an internal pressure of the sample well.

30. The pressure vessel of any one of claims 27 to 29, wherein the overflow channel is adapted to receive a temperature sensor.
